(19) United States
(12) Patent Application Publication
(10) Pub. No.: US 2006/0084099 A1
(43) Pub. Date: Apr. 20, 2006

(54) Product identification method

(75) Inventors: Hiroshi Yokoyama, Kanagawa (JP); Masahiko Yamanaka, Kanagawa (JP); Kentarou Watanabe, Kanagawa (JP); Tsunehiko Higuchi, Nagoya (JP); Hidetoshi Hayashi, Nagoya (JP)

Correspondence Address: FOLEY AND LARDNER LLP;SUITE 500, 3000 K STREET NW, WASHINGTON, DC, 20007, US

(73) Assignees: NISSAN MOTOR CO., LTD.; Tsunehiko HIGUCHI

(21) Appl. No.: 11/237,884

(22) Filed: Sep. 29, 2005

(30) Foreign Application Priority Data
Sep. 30, 2004 (JP) 2004-286558
Aug. 26, 2005 (JP) 2005-245807

(51) Int. Cl. C12Q 1/68; G06F 19/00; C12P 19/34

(52) U.S. Cl. 435/6; 702/20; 435/91.2

(57) ABSTRACT

A product identification method of one embodiment of the invention includes the steps of providing a product with an information nucleic acid having a segment of known base sequence randomly selected for the product, extracting the information nucleic acid from the product, amplifying the information nucleic acid by a polymerase chain reaction; and determining a base sequence of the information nucleic acid to identify the product by the base sequence of the information nucleic acid.

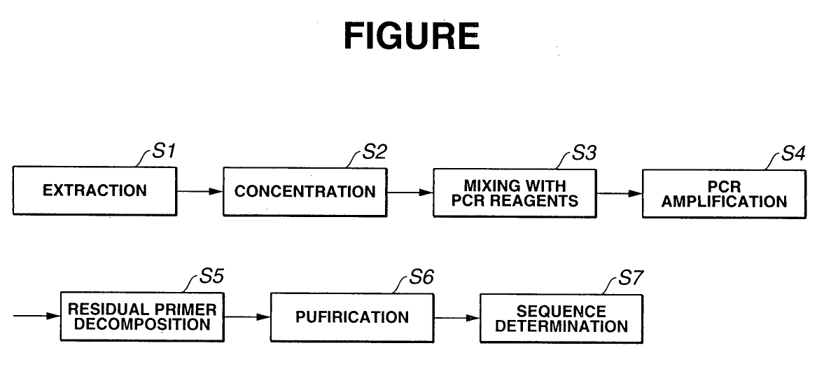

### BACKGROUND OF THE INVENTION

The present invention relates to a method for identifying a product by an information nucleic acid.

Various individual identification mediums such as registration plates, watermarks in banknotes, IC chips and photographic prints on credit cards have been widely used. There is a drawback that these identification mediums can be removed by exfoliation, cutting and erasing etc. For this reason, the development of unremovable and indelible identification mediums is desired.

On the other hand, nucleic acids such as deoxyribonucleic acids (DNA) appear ubiquitous in all organisms on Earth, and every living cell contains at least one DNA molecule as an informational biomolecule to carry genetic information. Many of DNA molecules have sequences corresponding to the amino acid sequences of proteins. Namely, DNA is a polynucleotide consisting of nucleotide units: deoxyadenosine (dA), deoxyguanosine (dG), deoxycytosine (dC) and thymidine (dT) polymerized directionally by phosphoric ester linkages. For a DNA molecule having n bases, the number of possible base sequences will be 4ⁿ. There will be about 4.3 billions of possible base sequences for a DNA molecule having 16 bases. Nowadays, DNA molecules having several tens of bases can be synthetically formed in any sequences. The base sequences of DNA molecules can also be determined automatically by means of a sequencer when the DNA molecules are present in certain amounts or more.

In view of the foregoing, Japanese Laid-Open Patent Publication No. 2004-159502 proposes a technique for mixing a nucleic acid with a water insoluble medium and applying the thus-obtained water insoluble medium to a product so as to label the product with the nucleic acid and judge the authentication of the product by detection of the nucleic acid.

### SUMMARY OF THE INVENTION

In the above-proposed technique, however, the authentication of the product is merely judged based on whether or not the nucleic acid can be amplified by a polymerase chain reaction (PCR). There is no description and no suggestion to authenticate a product based on the presence or absence of a nucleic acid in the product and to identify products individually by nucleic acid sequences even when the products are of the same production line.

Further, it is required, in a case where a personal property such as a vehicle is stolen or damaged, to make an early identification of the target object by any trace e.g. material pieces left in the scene even when the perpetrator runs away from the scene.

It is therefore an object of the present invention to provide a method for identifying a product using an information nucleic acid as an individual identification medium indicative of individual source and history information of the product.

According to one aspect of the present invention, there is provided a product identification method, comprising: providing a product with an information nucleic acid having a segment of known base sequence randomly selected for the product; extracting the information nucleic acid from the product; amplifying the information nucleic acid by a polymerase chain reaction; and determining a base sequence of the information nucleic acid to identify the product by the base sequence of the information nucleic acid.

According to another aspect of the present invention, there is provided a method for identifying a product, the product being provided with an information nucleic acid having a segment of known base sequence randomly selected for the product, the method comprising: extracting the information nucleic acid from the product; amplifying the information nucleic acid by a polymerase chain reaction; and determining a base sequence of the information nucleic acid to identify the product by the base sequence of the information nucleic acid.

According to still another embodiment of the present invention, there is provided a product identification method, comprising: selecting information nucleic acids having segments of different and known base sequences; providing products with the information nucleic acids, respectively; taking any one of the products as a sample; extracting the information nucleic acid from the any one of the products; amplifying the information nucleic acid by a polymerase chain reaction; and determining a base sequence of the extracted and amplified information nucleic acid to identify the any one of the products by the determined nucleic acid sequence.

The other objects and features of the present invention will also become understood from the following description.

### DESCRIPTION OF THE DRAWING AND SEQUENCE LISTING

FIGURE is a flowchart for a product identification procedure according to one exemplary embodiment of the present invention.

SEQ ID NO. 1 is an information DNA sequence used in the example.

SEQ ID NO. 2 is an identifiable DNA sequence used in the example.

SEQ ID NO. 3 and NO. 4 are primer sequences used in the example.

### DESCRIPTION OF THE EMBODIMENTS

The present invention will be described below in detail.

A product identification method of the present embodiment includes the steps of providing a product with an information nucleic acid having a segment of known and randomly selected base sequence, extracting the nucleic acid from the product, amplifying the nucleic acid by a polymerase chain reaction (PCR), and then, determining a base sequence of the nucleic acid for individual identification of the product. A trace amount of information nucleic acid can be easily introduced into a product or a material thereof, but cannot be easily removed from the product. It is thus possible in the present embodiment to identify the specific individual information (such as source and history) of the product by detecting such a trace amount of information nucleic acid from the product. It is also possible in the present embodiment to identify mass-produced products individually and distinguish the products from one another, even when the products are of the same production line, by selecting information nucleic acids of different known base sequences and then labeling the products with the selected information nucleic acids, respectively.

Herein, the “known” base sequence of the information nucleic acid means that the base sequence of the information nucleic acid is already known, and the “randomly selected” base sequence of the information nucleic acid means that the base sequence of the information nucleic acid is randomly selected for the product from any detectable (identifiable) nucleic acid sequences.

Specific examples of the information nucleic acid usable in the present embodiment are deoxyribonucleic acids (DNA), ribonucleic acids (RNA) and derivatives thereof. The information nucleic acid can be either natural or artificial. In view of the stability of the information nucleic acid in extreme usage conditions of the product, it is desirable that the information nucleic acid is artificial. In this case, it is also advantageous in that the artificial nucleic acid can attain a base sequence that does not occur in the natural nucleic acid.

The size of the information nucleic acid is not particularly restricted. The number of bases in the information nucleic acid molecule is preferably 200 or less, more preferably about 100. When the base number of the information nucleic acid exceeds 200, it is likely that unreacted segments occur gradually during nucleic acid preparation so that the defective sequence content of the information nucleic acid becomes increased.

It is desirable that thymines are not adjacent to each other in the base sequence of the information nucleic acid in order to prevent the thymines from being dimerized together.

It is also desirable that at least one of a hydroxyl group on the 5′-end and a hydroxyl group on the 3′-end of the information nucleic acid is modified with a protective group in view of the stability of the nucleic acid in the combined use of the nucleic acid with a hydroxyl-reactive compound and in extreme usage conditions of the product. Specific examples of the protective group usable in the present embodiment include phosphoric ester groups, acyl groups, alkoxycarbonyl groups, benzyl groups, substituted benzyl groups and aryl groups. For example, a natural DNA molecule having the following chemical formula (I) can be modified into a 5′-end modified derivative having the following chemical formula (II) although both of them are usable as the information nucleic acid: 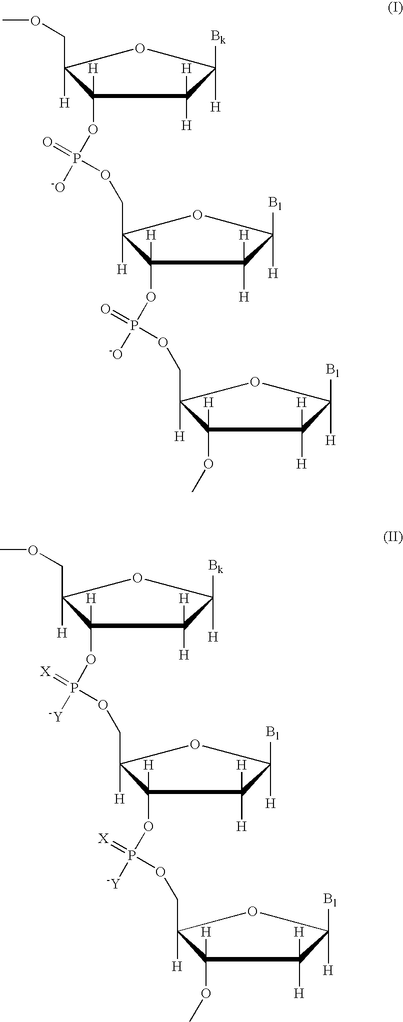
where X represents oxygen and Y represents sulfur for DNA of the phosphorothioate type; and both of X and Y represent sulfur for DNA of the phosphorodithioate type.

In the case of using RNA as the information nucleic acid, a hydroxyl group on the 2′-end of the RNA molecule may alternatively be modified with the above-specified protective group.

The stabilization of the information nucleic acid with the protective group or groups allows easy and precise nucleic acid detection in the product identification method.

For identification of the product, the information nucleic acid is first extracted from the product. The process of extracting the information nucleic acid from the product is not particularly restricted. For example, the information nucleic acid can be eluted from the product with water.

Next, the information nucleic acid is amplified by a PCR process.

It is a known fact that, in a PCR process, PCR primers bind or anneal to a nucleic acid at their complementary sites. Namely, the nucleic acid has to include sites to which the PCR primers are complementary (hereinafter referred to as “primer binding sites”) in order for the nucleic acid to be amplified by the PCR process. In the present embodiment, the primer binding sites correspond to both ends of the base sequence of the information nucleic acid or nearly the whole of the base sequence of the information nucleic acid.

If the primer binding sites are not preset, it becomes necessary to prepare various kinds of PCR primers for any possible nucleic acid sequences and to secure a large amount of test sample. This results in inefficient PCR amplification of the nucleic acid. Thus, the primer binding sites of the information nucleic acid may be optionally limited to some preset patterns in order to reduce the number of kinds of PCR primers to be prepared, amplify the nucleic acid by the PCR process in an efficient manner and identify the product in a shorter time.

The base number of each primer binding site of the information nucleic acid is preferably 5 or greater, more preferably 10 or greater. When the base number of the primer binding sites of the information nucleic acid is less than 5, the number of possible identifiable sequences of the information nucleic acid decreases so that it takes much time to identify a plurality of products individually. The base number of each primer binding site of the information nucleic acid is also preferably 100 or less. When the base number of the primer binding sites of the information nucleic acid exceeds 100, the content of the PCR by-product component of defective sequence becomes so high that the purification of the target PCR product component is burdensome and, in some cases, extremely difficult. In view of the efficiency of the PCR process, the base number of the primer binding sites of the information nucleic acid is most preferably 10 to 30.

In the case of using RNA as the information nucleic acid, it is possible to amplify RNA by a so-called RT-PCR process, i.e., by reverse transcribing a RNA molecule with a reverse transcriptase to obtain a DNA molecule having a sequence complementary to the initial RNA molecule, and then, subjecting the obtained DNA molecule to a polymerase chain reaction.

It is particularly desirable that the information nucleic acid includes primer binding sites at both ends thereof and an identification information site at a location in between the primer binding sites in order to set more specific information for individual identification of the product. In the case of using DNA as the information nucleic acid, for example, a strand of DNA may include a first primer binding site having a sequence of bases X₁ to Xₗ at one terminal thereof, a second primer binding site having a sequence of bases P₁ to Pₙ at the other terminal thereof and an identification information site having a sequence of bases B₁ to Bₘ between the first and second primer binding sites as expressed by the following formula (III): 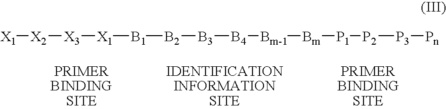
where X₁ to Xₗ, P₁ to Pₙ and B₁ to Bₘ independently represent any of deoxyadenosine (dA), deoxyguanosine (dG), deoxycytosine (dC) and thymidine (dT). Both of the above DNA strand or the helical complex of the above DNA strand and the other DNA strand complementary thereto are usable as the information nucleic acid. (Namely, the information nucleic acid can be either single-stranded or double-stranded.) The base sequences of the primer binding sites are preferably designed to stabilize the linkages between the PCR primers and the primer binding sites and to replicate the information nucleic acid by the PCR process smoothly.

More specifically, the PCR process is performed by mixing an eluate solution containing therein the information nucleic acid with various PCR reagents such as a PCR buffer, a sterile purified water, at least one kind of PCR primer, 2,3-dideoxyribonucleoside triphosphate (dNTP) and a polymerase, and then, subjecting the thus-obtained nucleic acid solution to a predetermined heating operation. In order to enhance the flexibility of the base sequence of the information nucleic acid, it is desirable to use two kinds of PCR primers. It also is desirable to use an artificial polymerase, in view of the improvements in efficiency and accuracy of the PCR process, when the information nucleic acid is artificial. The kind of the artificial polymerase is not particularly restricted. As the artificial polymerase, there may be used a HIV-1 (Human Immunodeficiency Virus 1) reverse transcriptase or an amino acid variant thereof.

The predetermined heating operation of the PCR process may desirably includes (1) heating the nucleic acid solution at 92 to 95° C. for 2 to 5 minutes, (2) repeating 20 to 50 cycles of (a) heating the nucleic acid solution at 92 to 95° C. for 30 to 60 seconds, (b) heating the nucleic acid solution at 20 to 50° C. for 30 to 60 seconds and (c) heating the nucleic acid solution at 70 to 80° C. for 30 to 120 seconds, and then, (3) heating the nucleic acid solution at 70 to 80° C. for 1 to 10 minutes.

It is particularly desirable that the nucleic acid solution is heated at 94° C. for 5 minutes in the heating treatment (1). When the nucleic acid solution is heated at 92° C. for less than 2 minutes in the heating treatment (1), the denaturation of the nucleic acid (the separation of the double-stranded nucleic acid into two nucleic acid strands) becomes difficult. By contrast, the enzyme becomes deactivated when the nucleic acid solution is heated at 95° C. for more than 5 minutes in the heating treatment (1). In the case that the information nucleic acid is single-stranded, the heating treatment (1) is not necessarily conducted.

In the heating treatment (a), it is particularly desirable that the nucleic acid solution is heated at 94° C. for 30 seconds. When the nucleic acid solution is heated at 92° C. for less than 30 seconds in the heating treatment (a), the rate of amplification of the nucleic acid becomes lowered. When the nucleic acid solution is heated at 95° C. for more than 60 seconds in the heating treatment (a), the enzyme becomes deactivated.

It is particularly desirable in the heating treatment (b) that the nucleic acid solution is heated at 40° C. for 30 seconds. When the nucleic acid solution is heated at 20° C. for less than 30 seconds in the heating treatment (b), the binding/annealing of the PCR primers to the primer binding sites of the nucleic acid becomes difficult. When the nucleic acid solution is heated at 50° C. for more than 60 seconds in the heating treatment (b), the enzyme becomes deactivated.

It is particularly desirable in the heating treatment (c) that the nucleic acid solution is heated at 72° C. for 30 seconds. When the nucleic acid solution is heated at 70° C. for less than 30 seconds in the heating treatment (c), the extension of the nucleic acid becomes insufficient. When the nucleic acid solution is heated at 80° C. for more than 120 seconds, the enzyme becomes deactivated.

It is particularly desirable that the nucleic acid solution is heated at 72° C. for 7 minutes in the heating treatment (3). When the nucleic acid solution is heated at 70° C. for less than 1 minute in the heating treatment (3), the extension of the nucleic acid becomes insufficient. It is a waste of time when the nucleic acid solution is heated at 80° C. for more than 10 minutes in the heating treatment (3).

It is further particularly desirable to repeat 30 thermal cycles of the denaturation phase (a), the annealing phase (b) and the extension phase (c). When the nucleic acid solution is subjected to less than 20 cycles of the denaturation phase (a), the annealing phase (b) and the extension phase (c), the rate of amplification of the nucleic acid becomes lowered. It is a waste of time when the nucleic acid solution is subjected to more than 50 cycles the denaturation phase (a), the annealing phase (b) and the extension phase (c).

After the PCR amplification of the information nucleic acid, the base sequence of the information nucleic acid is determined.

For prompt sequence determination and product identification, it is desirable to prepare a database storing therein a collection of data on various known kinds of information nucleic acids and refer the data on the information nucleic acid extracted from the product to the database. The data may be stored in terms of e.g. the time required for electrophoresis of the nucleic acid or the migration length of the nucleic acid in gel filtration (measured by feeding the nucleic acid into a control lane in a measurement unit).

In the present embodiment, the product can be any industrial product selected from the group consisting of paints, resins, oils and fats, fibers, fabrics, leather products, wood products, papers, prints, adhesives and combinations thereof.

Referring now to FIGURE, a product identification procedure will be explained below in more detail by taking as an example the case of using double-stranded information DNA as the information nucleic acid in the product.

At step S1, the information DNA is extracted from the product by e.g. cutting a test sample from the product, powdering the test sample and then mixing the sample powder with a small amount of water. Hydrolysis etc. may be performed for efficient extraction of the information DNA if the information DNA is supported on e.g. fine particles by chemical bonds in the product.

At step S2, the thus-obtained DNA solution is concentrated by lyophilization or centrifugal evaporation.

At step S3, the DNA solution is mixed with various PCR reagents including two kinds of primers and a polymerase.

At step S4, the DNA solution is subjected to the predetermined heating operation for PCR amplification of the extracted information DNA.

At step S5, residual primers are decomposed by treatment with e.g. a S1 nuclease (an enzyme capable of catalyzing the splitting of a DNA strand). Specific examples of the DNA strand splitting enzyme usable in the present embodiment include a Taq DNA polymerase, a Tth DNA polymerase, a Tfl DNA polymerase, a Vent DNA polymerase, a Pfu polymerase, a Bca BEST polymerase and a KOD DNA polymerase.

At step S6, the target reaction product component of the double-stranded information DNA is purified by gel filtration.

At step S7, the base sequence of the information DNA is determined by means of a sequencer. A mass spectroscope may be used solely in place of the sequencer or in combination with the sequencer to determine the base sequence of the information DNA.

The operation steps similar to steps S3 and S4 may be repeated between steps S6 and S7 for further amplification of the target DNA.

In view of the ease of isolation and purification of the information nucleic acid, it is further desirable that a hydroxyl group on the 5′-end of the information nucleic acid is modified with biotin or fluorescent molecule. Biotin combines with a specific protein: avidin so that the 5′-biotinylation of the nucleic acid allows selective absorption of the nucleic acid in an avidin column. By contrast, the fluorescent labeling of the nucleic acid allows precise detection and easy purification of the nucleic acid. The individual identification of the product becomes much easier by enhancing the ease of isolation and purification of the information nucleic acid. Further, the 5′-end of the information nucleic acid may be substituted with sulfur so that the 5′-end substituted nucleic acid can be readily isolated by eluting with water and feeding the eluate in a column of gold(Au)-coated carrier.

The present invention will be described in more detail by reference to the following example. However, it should be noted that the following example is only illustrative and not intended to limit the invention thereto.

### Sample Preparation

A clear paint composition was prepared by mixing a clear paint (available under the trade name of “SUPERLACK O-130 GN3” from NIPPON PAINT Co., Ltd.) with 5 μg of Information DNA of SEQ ID NO. 1 of the Sequence Listing (including Identifiable DNA of SEQ ID NO. 2 of the Sequence Listing) per 100 g of the clear paint and stirring the mixture for 1 hour.

A coating of cationic electropaint (available under the trade name “POWERTOP U600M” from NIPPON PAINT Co., Ltd.) was applied by electrodeposition to a dull-finished steel plate (treated with zinc phosphate and having a size of 70 mm×150 mm and a thickness of 0.8 mm) in such a manner that the cationic electropaint coating had a dry thickness of 20 μm. The electropaint-coated steel plate was baked at 160° C. for 30 minutes.

Subsequently, an intermediate coating (available under the trade name “HIGH-EPICO No. 500, Color: Gray” from NOF CORPORATION) was applied to the steel plate in such a manner that the intermediate coating had a dry thickness of 30 μm. The coated steel plate was baked at 140° C. for 1 hour.

The prepared clear paint composition was then applied to the steel plate in such a manner that the coating of the clear paint composition had a dry thickness of 30 μm. The coated steel plate was baked at 140° C. for 1 hour.

### Identification

A sample of the three-layered coating film was cut into pieces by a cutter and stirred in 5 ml of sterile purified water with a magnetic stirrer to obtain an eluate solution containing therein the information DNA.

The DNA solution were separated from the sample coating pieces by a centrifuge and concentrated by a centrifugal evaporator.

After that, 5 μl of the DNA solution was mixed with 5 μl of PCR buffer, 0.25 μl of Taq polymerase, 24. 75 μl of sterile purified water, two kinds of PCR primers: 5 μl of Primer 1 of SEQ ID NO. 3 of the Sequence Listing and 5 μl of Primer 2 of SEQ ID NO. 4 of the Sequence Listing and 5 μl of 2,3-dideoxyribonucleoside triphosphate (2 mM).

The DNA solution was heated at 94° C. for 5 minutes and subjected to 30 repeated cycles of heating at 94° C. for 30 seconds, at 40° C. for 30 seconds and at 72° C. for 30 seconds.

The DNA solution was then treated at 72° C. for 7 minutes and kept at 4° C.

The PCR primers remaining unreacted in the solution were decomposed by specific treatment with S1 nuclease.

The target double-stranded Information DNA was purified by gel filtration.

The purified DNA solution was mixed with one kind of PCR primer: Primer 1 of SEQ ID NO. 3 of the Sequence Listing and fluorescent-labeled 2,3-dideoxyribonucleoside triphosphate.

The DNA solution was again heated at 94° C. for 5 minutes and subjected to 30 repeated cycles of heating at 94° C. for 30 seconds, at 40° C. for 30 seconds and at 72° C. for 30 seconds.

The target double-stranded Information DNA was purified by gel filtration and put through an automatic sequencer to determine the sequence of the Information DNA.

As a result, the Information DNA originally incorporated into the clear coating composition was detected successfully.

As described above, it is therefore possible according to the present embodiment to make an individual identification of the product by the base sequence of the information nucleic acid extracted from the product.

The entire contents of Japanese Patent Application No. 2004-286558 (filed on Sep. 30, 2004) and No. 2005-245807 (filed on Aug. 26, 2005) are herein incorporated by reference.

Although the present invention has been described with reference to a specific embodiment of the invention, the invention is not limited to the above-described embodiment. Various modification and variation of the embodiment described above will occur to those skilled in the art in light of the above teaching. The scope of the invention is defined with reference to the following claims.

What is claimed is:

1. A product identification method, comprising:
providing a product with an information nucleic acid having a segment of known base sequence randomly selected for the product;
extracting the information nucleic acid from the product;
amplifying the information nucleic acid by a polymerase chain reaction; and
determining a base sequence of the information nucleic acid to identify the product by the base sequence of the information nucleic acid.

2. The product identification method of claim 1, wherein said determining includes referring data on the information nucleic acid to a database.

3. The product identification method of claim 1, wherein the base sequence of the information nucleic acid includes primer binding sites at least at both ends thereof, and each of the primer binding sites contains therein 1 to 30 bases.

4. The product identification method of claim 1, wherein said amplifying includes:
mixing a solution containing therein the extracted information nucleic acid with a polymerase chain reaction buffer, sterile purified water, at least one kind of polymerase chain reaction primer, 2,3-dideoxyribonucleoside triphosphate and a polymerase; and
subjecting the solution to a predetermined heating operation.

5. The product identification method of claim 4, wherein the predetermined heating operation includes:
(1) heating the solution at 92 to 95° C. for 2 to 5 minutes;
(2) repeating 20 to 50 cycles of (a) heating the solution at 92 to 95° C. for 30 to 60 seconds, (b) heating the solution at 20 to 50° C. for 30 to 60 seconds and (c) heating the solution at 70 to 80° C. for 30 to 120 seconds; and then
(3) heating the solution at 70 to 80° C. for 1 to 10 minutes.

6. The product identification method of claim 4, wherein the information nucleic acid is an artificial nucleic acid and the polymerase is an artificial polymerase.

7. The product identification method of claim 6, wherein the artificial polymerase is either a HIV-1 reverse transcriptase or an amino acid variant thereof.

8. The product identification method of claim 4, wherein two kinds of polymerase chain reaction primers are mixed into the solution.

9. The product identification method for claim 1, further comprising: labeling the information nucleic acid with a fluorescent molecule or biotin.

10. The product identification method of claim 1, wherein the product is selected from the group consisting of paints, resins, oils and fats, fibers, fabrics, leather products, wood products, papers, prints, adhesives and combinations thereof.

11. A method for identifying a product, the product being provided with an information nucleic acid having a segment of known base sequence randomly selected for the product, the method comprising:
extracting the information nucleic acid from the product;
amplifying the information nucleic acid by a polymerase chain reaction; and
determining the base sequence of the information nucleic acid to identify the product by the base sequence of the information nucleic acid.

12. The method of claim 11, wherein said determining includes referring data on the information nucleic acid to a database.

13. The method of claim 11, wherein the base sequence of the information nucleic acid includes primer binding sites at least at both ends thereof, and each of the primary binding sites contains therein 10 to 30 bases.

14. The method of claim 11, wherein said amplifying includes:
mixing a solution containing therein the extracted information nucleic acid with a polymerase chain reaction buffer, sterile purified water, at least one kind of polymerase chain reaction primer, 2,3-dideoxyribonucleoside triphosphate and a polymerase; and then
subjecting the solution to a predetermined heating operation.

15. The method of claim 14, wherein the predetermined heating operation includes:
(1) heating the solution at 92 to 95° C. for 2 to 5 minutes;
(2) repeating 20 to 50 cycles of (a) heating the solution at 92 to 95° C. for 30 to 60 seconds, (b) heating the solution at 20 to 50° C. for 30 to 60 seconds and (c) heating the solution at 70 to 80° C. for 30 to 120 seconds; and then
(3) heating the solution at 70 to 80° C. for 1 to 10 minutes.

16. The method of claim 14, wherein the information nucleic acid is an artificial nucleic acid, and the polymerase is an artificial polymerase.

17. The method of claim 11, wherein the product is selected from the group consisting of paints, resins, oils and fats, fibers, fabrics, leather products, wood products, papers, prints, adhesives and combinations thereof.

18. A product identification method, comprising:
selecting information nucleic acids having segments of different and known base sequences;
providing products with the information nucleic acids, respectively;
taking any one of the products as a sample;
extracting the information nucleic acid from said any one of the products;
amplifying the information nucleic acid by a polymerase chain reaction;
determining a base sequence of the extracted and amplified information nucleic acid to identify said any one of products by the determined nucleic acid sequence.

19. The product identification method of claim 18, wherein said determining includes referring data on the information nucleic acid to a database.

20. The product identification method of claim 18, wherein the base sequence of the information nucleic acid includes primer binding sites at least at both ends thereof, and each of the primer binding sites contains therein 10 to 30 bases.

21. The product identification method of claim 18, wherein said amplifying includes:
mixing a solution containing therein the extracted information nucleic acid with a polymerase chain reaction buffer, sterile purified water, at least one kind of polymerase chain reaction primer, 2,3-dideoxyribonucleoside triphosphate and a polymerase; and
subjecting the solution to a predetermined heating operation.

22. The product identification method of claim 21, wherein the predetermined heating operation includes:
(1) heating the solution at 92 to 95° C. for 2 to 5 minutes;
(2) repeating 20 to 50 cycles of (a) heating the solution at 92 to 95° C. for 30 to 60 seconds, (b) heating the solution at 20 to 50° C. for 30 to 60 seconds and (c) heating the solution at 70 to 80° C. for 30 to 120 seconds; and then
(3) heating the solution at 70 to 80° C. for 1 to 10 minutes.

23. The product identification method of claim 21, wherein the information nucleic acid is an artificial nucleic acid, and the polymerase is an artificial polymerase.

24. The product identification method of claim 18, wherein the product is selected from the group consisting of paints, resins, oils and fats, fibers, fabrics, leather products, wood products, papers, prints, adhesives and combinations thereof.


SEQUENCE LISTING (4 sequences)

SEQ ID NO: 1 - DNA, 51 bp, Artificial Sequence
  Description of Artificial Sequence Synthetic oligonucleotide
tgcacgcaccgtgtactcgggattaattggaggagtggacacgttggtcgg

SEQ ID NO: 2 - DNA, 15 bp, Artificial Sequence
  Description of Artificial Sequence Synthetic oligonucleotide
gggattaattggagg

SEQ ID NO: 3 - DNA, 18 bp, Artificial Sequence
  Description of Artificial Sequence Synthetic primer
tgcacgcaccgtgtactc

SEQ ID NO: 4 - DNA, 18 bp, Artificial Sequence
  Description of Artificial Sequence Synthetic primer
ccgaccaacgtgtccact